(19) United States
(12) Patent Application Publication
(10) Pub. No.: US 2018/0150690 A1
(43) Pub. Date: May 31, 2018

(54) VIRTUAL REALITY DEVICE USING EYE PHYSIOLOGICAL CHARACTERISTICS FOR USER IDENTITY AUTHENTICATION

(71) Applicant: Alibaba Group Holding Limited, George Town (KY)

(72) Inventor: Huanmi Yin, Hangzhou (CN)

(73) Assignee: Alibaba Group Holding Limited, George Town (KY)

(21) Appl. No.: 15/819,034

(22) Filed: Nov. 21, 2017

(30) Foreign Application Priority Data
Nov. 29, 2016 (CN) 201621293788.0

(51) Int. Cl. G06K 9/00; A61B 3/14; A61B 3/00; H04N 5/225; G06T 19/00

(52) U.S. Cl. CPC G06K 9/00617; A61B 3/14; A61B 3/0008; H04N 5/33; H04N 5/2256; G06T 19/006; G06K 9/00604

(57) ABSTRACT

In an implementation, a virtual reality (VR) device includes a housing that has two openings. Each of the two openings hosts a camera lens and a nose groove. The VR device also includes one or more cameras distributed around each of the camera lenses for capturing one or more eye physiological characteristics of a VR device user.

This application claims priority to Chinese Patent Application No. 201621293788.0, filed on Nov. 29, 2016, which is incorporated by reference in its entirety. The subject matter of the present invention is also related to U.S. patent application Ser. No. ______, filed on ______, which is incorporated by reference in its entirety.

### TECHNICAL FIELD

This disclosure relates to user identity authentication, and more particularly to user identity authentication based on eye physiological characteristics.

### BACKGROUND

Virtual reality (VR) is a computer technology that uses VR devices, such as headsets, sometimes in combination with physical spaces or multi-projected environments, to generate realistic images, sounds, and other sensations that simulate a user's physical presence in a three dimensional (3D) virtual environment and allow the VR user to interact with the virtual environment.

With the evolvement of VR hardware, VR technologies have been increasingly used in areas, including architecture, health care, education, entertainment, gaming, and online shopping. Many applications, such as those for gaming, content consumption, and productivity, have been developed to provide user an immersive experience using VR technology. Many VR applications allow for operations, including in-application purchases, user customization, or parental control. These operations should require user identity authentication for security purposes.

### SUMMARY

The present disclosure describes methods and apparatus, including computer-implemented methods, computer program products, and computer systems for service control and user identity authentication based on virtual reality (VR).

In an implementation, a VR device includes a housing that has two openings. Each of the two openings hosts a camera lens and a nose groove. The VR device also includes one or more cameras distributed around each of the camera lenses for capturing one or more eye physiological characteristics of a VR device user.

The previously described implementation is implementable using a computer-implemented method; a non-transitory, computer-readable medium storing computer-readable instructions to perform the computer-implemented method; and a computer-implemented system comprising a computer memory interoperably coupled with a hardware processor configured to perform the computer-implemented method/the instructions stored on the non-transitory, computer-readable medium.

The subject matter described in this specification can be implemented in particular implementations, so as to realize efficient user identity authentications in a virtual environment to provide faster, more convenient operations, and a more immersive experience to VR users.

The details of one or more implementations of the subject matter of this specification are set forth in the Detailed Description, the Claims, and the accompanying drawings. Other features, aspects, and advantages of the subject matter will become apparent to those of ordinary skill in the art from the Detailed Description, the Claims, and the accompanying drawings.

### DESCRIPTION OF DRAWINGS

FIG. 1A is a schematic diagram illustrating an example structure of a virtual reality device with eye physiological characteristics sensing capability, according to an implementation of the present disclosure.

FIG. 1B is a schematic diagram illustrating an example camera structure of the virtual reality device with eye physiological characteristics sensing capability, according to an implementation of the present disclosure.

FIG. 2 is a flowchart illustrating an example of a method for performing user identity authentication based on eye physiological characteristics eye physiological characteristics in a virtual reality scenario, according to an implementation of the present disclosure

FIG. 3 is a block diagram illustrating an example of a computer-implemented system used to provide computational functionalities associated with described algorithms, methods, functions, processes, flows, and procedures, according to an implementation of the present disclosure.

Like reference numbers and designations in the various drawings indicate like elements.

### DETAILED DESCRIPTION

The following detailed description describes technologies related to virtual reality (VR) hardware for capturing a user's eye physiological characteristics (EPC), and is presented to enable any person skilled in the art to make and use the disclosed subject matter in the context of one or more particular implementations. Various modifications, alterations, and permutations of the disclosed implementations can be made and will be readily apparent to those or ordinary skill in the art, and the general principles defined can be applied to other implementations and applications, without departing from the scope of the present disclosure. In some instances, one or more technical details that are unnecessary to obtain an understanding of the described subject matter and that are within the skill of one of ordinary skill in the art may be omitted so as to not obscure one or more described implementations. The present disclosure is not intended to be limited to the described or illustrated implementations, but to be accorded the widest scope consistent with the described principles and features.

VR is a computer technology that uses VR devices, such as headsets, sometimes in combination with physical spaces or multi-projected environments, to generate realistic images, sounds, and other sensations that simulate a user's physical presence in a three-dimensional (3D) virtual environment and allow the VR user to interact with the virtual environment.

With the evolvement of VR hardware, VR technologies have been increasingly used in areas, including architecture, health care, education, entertainment, gaming, and online shopping. Many applications, such as those for gaming, content consumption, and productivity, have been developed to provide the user with an immersive experience using VR technology. Many VR applications allow for operations, including in-application purchases, user customization, or parental control. These operations should require user identity authentication for security purposes.

The present disclosure describes VR device technologies that can perform biometric authentication based on the user's EPC. The VR device can be used to perform VR applications or software developed based on VR technologies. The VR applications can provide the VR device user with 3D immersive VR experience.

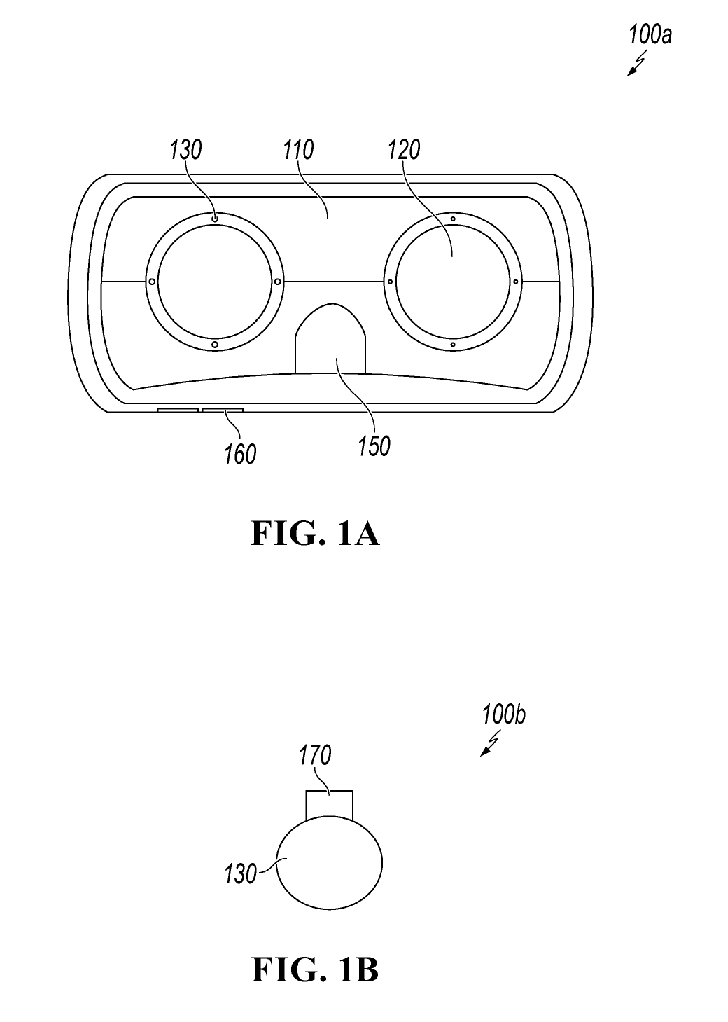

FIG. 1 is a schematic diagram illustrating an example structure of a VR device 100a with EPC sensing capability, according to an implementation of the present disclosure. In some implementations, the VR device can be a VR headset which can be worn by a VR user on the VR user's head. The VR device 100a can include hardware components that can detect and capture EPC of the VR user for user identity authentication. Example EPC can include iris characteristics and eye print characteristics.

The illustrated VR device 100a includes a housing 110. In some implementations, the housing 110 can have two openings corresponding to relative positions of the VR user's eyes. The two openings can each host a lens 120. In some cases, the lenses 120 can include a lens assembly that can convert light that passes through the openings into visual effects shown to the VR user. One or more cameras 130 can be distributed around each of the two lenses 120 for capturing the EPC of the user. In some cases and as illustrated, the cameras 130 can be uniformly distributed around the lens 120. In other cases, the lenses can be grouped into particular positions (for example, on one side of the opening). In some cases, the cameras 130 can be positioned to ensure that the lenses 120 are located between the VR user's eyes and the cameras 130. In some cases, the cameras 130 can be positioned to capture images of the VR user's eyes from more than one direction to obtain more complete EPC data set. In some implementations, a required quantity of cameras 130 can be determined based on a quality requirement associated with the EPC.

The VR device 100a also includes a nose groove in-between and under the lenses 120 to accommodate the VR user's nose. As such, the VR device 100a can be fit more closely proximate to the VR user's eyes.

In some implementations, the VR device 100a can also include a data interface 160, such as a Universal Serial Bus (USB) port or a WIFI chip. The data interface 160 can be used to electrically or wirelessly connect the VR device 100a to an external user computing device, such as a smartphone, a tablet, or a computer.

In some implementations, the external user computing device can acquire image from the cameras 130 or other data from the VR device 100a through a USB or other cable. In some cases, the external user computing device can acquire data from the VR device 100a through wireless connections, such as WIFI or BLUETOOTH. In some cases, the data interface 160 can also be used to charge or supply power to the VR device 100a.

Referring to FIG. 1B, FIG. 1B is a schematic diagram illustrating an example camera structure 100b of the virtual reality device 100a with eye physiological characteristics sensing capability, according to an implementation of the present disclosure. The camera 130 shown in FIG. 1B can be any of the cameras 130 used in the VR device 100a illustrated in FIG. 1A. As shown in FIG. 1B, a light emitting diode (LED) light 170 can be attached to the camera 130. In some cases, the LED light 170 can have a one-to-one relationship with the camera 130. In some cases, more than one LED light 170 can be used for each camera 130. The LED light 170 can be used to compensate for poor light conditions inside the enclosure of the housing 110 to improve image quality of images acquired and returned by the camera 130.

The EPC can include eyeprint characteristics, iris characteristics, a combination of eyeprint characteristics and iris characteristics, or other eye physiological characteristics. The eyeprint characteristics can be the texture of the sclera, which is the white part of the eye. Each individual person's sclera texture is unique. The iris is the annular portion between the pupil and the sclera, which also has unique texture for each individual.

In some cases, eyeprint characteristics are used as the EPC for user identity authentication, the camera 130 used can be an RGB camera. The LED lights 170 can be used to make one-to-one compensation for the light of the RGB camera 130. In some cases, iris characteristics are used as EPC for user identity authentication. For example, the cameras 130 distributed around the lenses 120 can be infrared cameras and the user's iris can be imaged under an emitted infrared LED light source. The infrared LED light source can provide one-to-one light compensation to the infrared cameras. In some cases, the VR device 100a can collect iris and eyeprint characteristics at the same time. In such cases, the cameras 130 can include both RGB cameras and infrared cameras. The LED lights 170 can include both infrared lights and regular LED lights.

The VR device 100a can also include a light sensor to detect the brightness of the ambient light incident on the lenses 120 of the VR device 100a. If the brightness is lower than a predetermined value and that can negatively affect capture of the EPC, the VR device 100a can turn on one or more of the LED lights 170 to perform one-to-one light compensation to the one or more cameras 130.

In some cases, the physical dimension of the VR device 100a can result in an insufficient depth-of-field between the cameras 130 and the VR user's eyes, such that the cameras 130 cannot be accurately focused on the eye of the VR user. In such cases, one or more macro lens cameras can be used. Alternatively, to adaptively adjust the focal length, one or more additional optical lenses can be installed at a position between the user's eyes and the lenses 120 to reflect or refract light rays coming from the user's eyes based on the internal shape of the VR device 100a (for example, the cavity of a VR headset). In some cases, special lenses such as those used in an endoscope can be used to collect the user's iris or eyeprint characteristics. In some cases, a user can manually adjust the focal length of the cameras 130 to obtain accurate iris or eyeprint images. In some cases, a dedicated summing hardware or software can be used to automatically adjust the focus of the cameras.

In some cases, the VR device 100a can communicate to a server through the data interface 160 for user identity authentication. The VR device 100a can transmit the collected EPC samples to the server though wired or wireless connections. The collected EPC samples can be compared with EPC samples pre-stored on the server. In some cases, the user can use the VR device 100a to obtain one or more EPC samples during service account registration. The one or more EPC samples can be saved locally for applications that allows offline biometric authentication or sent to a service server for applications that require online authentication. For example, when the service is a VR payment service, the service account of the user can be a payment account. The VR device 100a can use an integrated eye sensor to obtain the user's EPC sample during payment account registration and save the sample locally or on the payment server. The EPC sample is associated with the user's payment account.

In some cases, the user can log-in to the user account for using the VR device 100a. The VR device 100a can send the EPC sample and VR device 100a user account log-in information as a registration message to the service server. The server can use the received information to associate the user's EPC sample to its service account and save the eye physiologic characteristics sample to a biometric characteristics database. In some cases, the user account can be the same as the service account.

In some cases, after the EPC sample is collected by the VR device 100a, the VR device 100a can generate a biometric recognition request to the service server. The biometric recognition request can include the user's user or service account information and EPC obtained by the eye sensor. The service server can provide a biometric recognition interface to the VR device 100a for receiving the request, and submit the request to the server. In some cases, the service server can cooperate with a biometric recognition server and the biometric recognition interface can be provided by the biometric recognition server.

The VR device 100a user can be authenticated if the one or more EPC matches at least a portion of the one or more pre-stored EPC samples. After receiving the biometric recognition request, the service server can parse the request, acquire the EPC and the user account information, and compare the EPC with EPC samples stored in a biometric characteristic database. In some cases, if the EPC matches at least a portion of the EPC samples, the service server can further verify whether the received user account information matches the account associated with the matching biometric sample. After comparing the biometric information and user account information with the corresponding information stored in the biometric characteristic database, the service server can return an authentication result to the VR device 100a. The authentication result can be returned as a Boolean-type return value (that is, true or false). If one or both the biometric information and user account information match the corresponding information stored in the biometric characteristic database, a “true” value can be returned to indicate that the biometric authentication succeeded. Otherwise, a “false” value can be returned to indicate that the biometric authentication failed.

In some cases, the VR device 100a can perform biometric authentication locally. The user's EPC can be saved in the VR device 100a during biometric information registration (for example, registering eyeprint information to the user account on the VR device 100a). After the user initiates the service in the VR scenario, the VR device 100a can collect the EPC of the user, and compare the EPC with the EPC samples saved during biometric information registration. If the EPC matches at least a portion of the saved information, the biometric authentication for the service succeeds. Otherwise, the biometric authentication fails.

In some cases, the VR device 100a can perform additional biometric authentication before sending the registration message to the service server for enhanced security. For example, the VR device 100a can prompt the user to input a password or other security information to verify the user's identity before sending the user's registration message to the service server. This process can prevent unauthorized users from fraudulently registering her/his biometric information to bind with the authorized user's user account. After service registration, the VR device 100a can initiate biometric authentication based on the user's EPC sample after the user initiates the service.

In some cases, the VR device 100a can perform a bioassay process before collecting biometric information to ensure that the VR device 100a is currently used by a real person. This process can prevent unauthorized users from using eyeprint or iris images of the authorized user for biometrical authentication and illegally accessing the authorized user's service account. Example bioassay processes can include eye-blinking or heartbeat recognition.

After a real user is detected using the bioassay process, the VR device 100a can prompt the user to perform biometric authentication for accessing the service account. Using again a VR payment service for example, the VR device 100a can present a text prompt in the VR scenario such as “Please scan your iris for payment authentication.”

In some cases, a server is communicated with to perform the service. If the biometric authentication is successful (for example, a returned value is “true”), the VR device 100a can present the service interface corresponding to the service to gather data related to the service, establish a service request, submit the service request to the service server through a service access interface, and perform further interactions with the service server, if needed, to perform the service.

Using again the VR payment service for example, the VR device 100a can present a payment interface to gather payment related data, such as user name, order information, and price, and then generate a payment processing request, and send the payment processing request to the service server. The service server can process the request and complete the payment.

The following example uses VR shopping to illustrate how biometric authentication based on EPC can provide safer, faster, and simpler service experience to the user. It is assumed that the eye physiological characteristic used is the eyeprint. In some implementations, the service for the VR shopping can be a payment service such as ALIPAY, the virtual element can be a virtual button presented in the VR shopping scenario, and the server for the payment service can be a payment server such as the ALIPAY platform based on a server cluster.

The user can first log in to the VR device 100a using her user account or payment account, complete eyeprint registration on the VR device 100a to associate the user's eyeprint to the payment account. The user's account information and corresponding eyeprint information can be stored in a biometric characteristic database on a payment server. After eyeprint registration, the user can use their finger (that is, fingerprint) for biometric authentication in a VR environment.

When wearing the VR device 100a for VR shopping, the items for sale can be presented to the user in the VR scenario and the user can flip through the item list, select items, or add items to shopping cart using gestures or head movements. In some cases, a virtual button (that is, the virtual element) for checkout or pay can be provided when an item is selected or added to the user's shopping cart. The user can again use gesture or head movements to move the operation focus (for example, a cursor) to the virtual button and use a predetermined gesture or head movement to select the virtual button.

The payment service can be initiated after the service is initiated by the user's voice command. In some cases, The VR device 100a can perform a bioassay process to determine whether a real user of the VR device 100a is present. If so, the VR device 100a can prompt a message “Please keep your eyes open for payment authentication” in the VR scenario.

After EPC are collected, the VR device 100a can send a biometric authentication request including the collected eyeprint information and the user's user or payment account information to the payment server. The payment server can compare the eyeprint information with the eyeprint information stored in the biometric characteristic database during eyeprint registration. If the received eyeprint information matches the eyeprint associated with the user account stored in the database, and the payment account information is consistent with the stored user account information, the payment server can return a Boolean-type “true” value to the VR device 100a to indicate that the eyeprint authentication is successful.

After having acknowledged that the payment was successful, the VR device 100a can present a payment interface for the user to input payment data, such as user name, order information, and payment amount related to the payment service, generate, and send a payment processing request to the payment server. The payment server can process the payment and complete the purchase.

In some cases, an “authentication-free for small amount” payment process can be used to further facilitate payment speed. For example, when the user initiates the payment service using voice command, the VR device 100a can check the price of the items and determine whether the payment amount is less than a preset amount (for example, 20 USD). If the payment amount is less than the preset amount, the VR device 100a can directly generate and send the payment processing request to the payment server without biometric authentication. Otherwise, the user may need to perform biometric authentication before payment service can be used.

In some cases, the VR device 100a can be used for faster and more convenient user identity authentication based on a user's eye physiological characteristics. When a user uses the VR device 100a to perform a service, the VR device can identify user interaction operations with virtual elements rendered in the virtual reality scenario using one or more sensors. When it is identified that the user interaction operations match one or more pre-stored operations, the VR device can invoke biometric authentication based on the user's EPC for user identity verification. If the biometric authentication is successful, the user can perform a service interaction to complete the service. As such, the VR device 100a can collect a user's EPC to quickly perform biometric authentication in the VR environment, to simplify the user identity authentication procedure and ensure account security. For example, when using a payment service in a VR environment, biometric authentication through the VR device can allow faster user identity authentication, as compared to entering a password through complicated user interactions with a virtual keyboard.

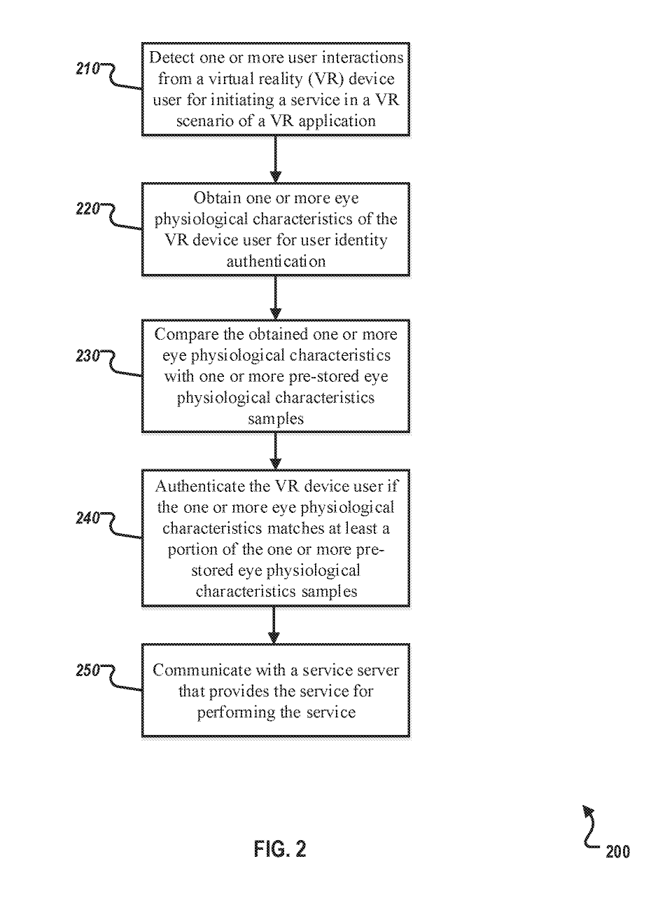

FIG. 2 is a flowchart illustrating an example of a method 200 for performing user identity authentication based on eye physiological characteristics eye physiological characteristics in a virtual reality scenario, according to an implementation of the present disclosure, according to an implementation of the present disclosure. For clarity of presentation, the description that follows generally describes method 200 in the context of the other figures in this description. However, it will be understood that method 200 can be performed, for example, by any system, environment, software, and hardware, or a combination of systems, environments, software, and hardware, as appropriate. In some cases, various steps of method 200 can be run in parallel, in combination, in loops, or in any order.

At 210, one or more user interactions from a VR device user for initiating a service in a VR scenario of a VR application are detected. The service can be any service or task offered in a VR application performed by the user that requires user identity authentication. In some cases, the service can be a local task performed by a user computing device or an online task performed by a server. For example, the service can be an online payment service, such as payment made in VR applications such as VR shopping, VR games, VR based video-on-demand, or donation to a VR live cast. Alternatively, the service can be a local service that requires user account login or password protected unlock. The VR application can be any software or application that is developed based on VR. The VR application can render a VR scenario through the VR device to provide user with immersive 3D experience. From 210, method 200 proceeds to 220.

At 220, one or more EPC of the VR device user are obtained for user identity authentication. After a service is initiated, the VR device can present a UI in a virtual scenario for user identity authentication. The VR device can be the VR device 100a discussed in the description of FIG. 1A that can include eye recognition function to facilitate user identity authentication. From 220, method 200 proceeds to 230.

At 230, the obtained one or more eye physiological characteristics are compared with one or more pre-stored eye physiological characteristics samples. In some cases, the VR device user can use the VR device such as the VR device 100a described in the description of FIG. 1A to obtain one or more EPC samples during service account registration. The one or more EPC samples can be saved locally for applications that allow offline biometric authentication or included in an authentication request to be sent to a service server for online authentication. From 230, method 200 proceeds to 240.

At 240, the VR device user is authenticated if the one or more EPC match at least a portion of the one or more pre-stored EPC samples. From 240, method 200 proceeds to 250.

At 250, a service server is communicated with to perform the service. If the biometric authentication is successful (for example, a returned value is “true”), the VR device can present the service interface corresponding to the service to gather data related to the service, establish a service request, submit the service request to the service server through a service access interface, and perform further interactions with the service server, if needed, to perform the service. After 250, method 200 stops.

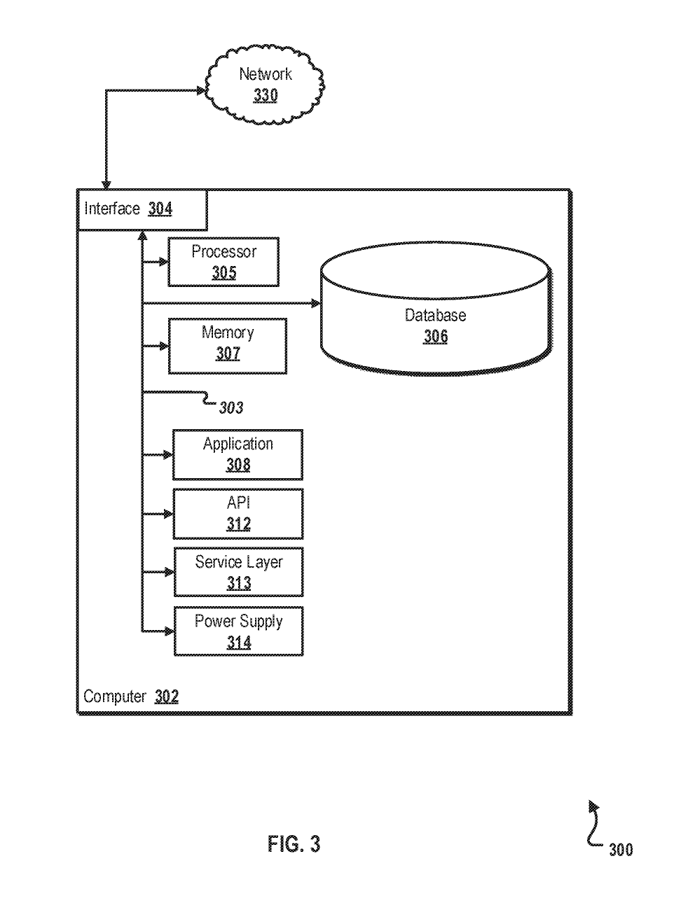

FIG. 3 is a block diagram illustrating an example of a computer-implemented System 300 used to provide computational functionalities associated with described algorithms, methods, functions, processes, flows, and procedures, according to an implementation of the present disclosure. In the illustrated implementation, System 300 includes a Computer 302 and a Network 330.

The illustrated Computer 302 is intended to encompass any computing device such as a server, desktop computer, laptop/notebook computer, wireless data port, smart phone, personal data assistant (PDA), tablet computer, one or more processors within these devices, another computing device, or a combination of computing devices, including physical or virtual instances of the computing device, or a combination of physical or virtual instances of the computing device. Additionally, the Computer 302 can include an input device, such as a keypad, keyboard, touch screen, another input device, or a combination of input devices that can accept user information, and an output device that conveys information associated with the operation of the Computer 302, including digital data, visual, audio, another type of information, or a combination of types of information, on a graphical-type user interface (UI) (or GUI) or other UI.

The Computer 302 can serve in a role in a distributed computing system as a client, network component, a server, a database or another persistency, another role, or a combination of roles for performing the subject matter described in the present disclosure. The illustrated Computer 302 is communicably coupled with a Network 330. In some implementations, one or more components of the Computer 302 can be configured to operate within an environment, including cloud-computing-based, local, global, another environment, or a combination of environments.

At a high level, the Computer 302 is an electronic computing device operable to receive, transmit, process, store, or manage data and information associated with the described subject matter. According to some implementations, the Computer 302 can also include or be communicably coupled with a server, including an application server, e-mail server, web server, caching server, streaming data server, another server, or a combination of servers.

The Computer 302 can receive requests over Network 330 (for example, from a client software application executing on another Computer 302) and respond to the received requests by processing the received requests using a software application or a combination of software applications. In addition, requests can also be sent to the Computer 302 from internal users (for example, from a command console or by another internal access method), external or third-parties, or other entities, individuals, systems, or computers.

Each of the components of the Computer 302 can communicate using a System Bus 303. In some implementations, any or all of the components of the Computer 302, including hardware, software, or a combination of hardware and software, can interface over the System Bus 303 using an application programming interface (API) 312, a Service Layer 313, or a combination of the API 312 and Service Layer 313. The API 312 can include specifications for routines, data structures, and object classes. The API 312 can be either computer-language independent or dependent and refer to a complete interface, a single function, or even a set of APIs. The Service Layer 313 provides software services to the Computer 302 or other components (whether illustrated or not) that are communicably coupled to the Computer 302. The functionality of the Computer 302 can be accessible for all service consumers using the Service Layer 313. Software services, such as those provided by the Service Layer 313, provide reusable, defined functionalities through a defined interface. For example, the interface can be software written in JAVA, C++, another computing language, or a combination of computing languages providing data in extensible markup language (XML) format, another format, or a combination of formats. While illustrated as an integrated component of the Computer 302, alternative implementations can illustrate the API 312 or the Service Layer 313 as stand-alone components in relation to other components of the Computer 302 or other components (whether illustrated or not) that are communicably coupled to the Computer 302. Moreover, any or all parts of the API 312 or the Service Layer 313 can be implemented as a child or a sub-module of another software module, enterprise application, or hardware module without departing from the scope of the present disclosure.

The Computer 302 includes an Interface 304. Although illustrated as a single Interface 304, two or more Interfaces 304 can be used according to particular needs, desires, or particular implementations of the Computer 302. The Interface 304 is used by the Computer 302 for communicating with another computing system (whether illustrated or not) that is communicatively linked to the Network 330 in a distributed environment. Generally, the Interface 304 is operable to communicate with the Network 330 and includes logic encoded in software, hardware, or a combination of software and hardware. More specifically, the Interface 304 can include software supporting one or more communication protocols associated with communications such that the Network 330 or hardware of Interface 304 is operable to communicate physical signals within and outside of the illustrated Computer 302.

The Computer 302 includes a Processor 305. Although illustrated as a single Processor 305, two or more Processors 305 can be used according to particular needs, desires, or particular implementations of the Computer 302. Generally, the Processor 305 executes instructions and manipulates data to perform the operations of the Computer 302 and any algorithms, methods, functions, processes, flows, and procedures as described in the present disclosure.

The Computer 302 also includes a Database 306 that can hold data for the Computer 302, another component communicatively linked to the Network 330 (whether illustrated or not), or a combination of the Computer 302 and another component. For example, Database 306 can be an in-memory, conventional, or another type of database storing data consistent with the present disclosure. In some implementations, Database 306 can be a combination of two or more different database types (for example, a hybrid in-memory and conventional database) according to particular needs, desires, or particular implementations of the Computer 302 and the described functionality. Although illustrated as a single Database 306, two or more databases of similar or differing types can be used according to particular needs, desires, or particular implementations of the Computer 302 and the described functionality. While Database 306 is illustrated as an integral component of the Computer 302, in alternative implementations, Database 306 can be external to the Computer 302.

The Computer 302 also includes a Memory 307 that can hold data for the Computer 302, another component or components communicatively linked to the Network 330 (whether illustrated or not), or a combination of the Computer 302 and another component. Memory 307 can store any data consistent with the present disclosure. In some implementations, Memory 307 can be a combination of two or more different types of memory (for example, a combination of semiconductor and magnetic storage) according to particular needs, desires, or particular implementations of the Computer 302 and the described functionality. Although illustrated as a single Memory 307, two or more Memories 307 or similar or differing types can be used according to particular needs, desires, or particular implementations of the Computer 302 and the described functionality. While Memory 307 is illustrated as an integral component of the Computer 302, in alternative implementations, Memory 307 can be external to the Computer 302.

The Application 308 is an algorithmic software engine providing functionality according to particular needs, desires, or particular implementations of the Computer 302, particularly with respect to functionality described in the present disclosure. For example, Application 308 can serve as one or more components, modules, or applications. Further, although illustrated as a single Application 308, the Application 308 can be implemented as multiple Applications 308 on the Computer 302. In addition, although illustrated as integral to the Computer 302, in alternative implementations, the Application 308 can be external to the Computer 302.

The Computer 302 can also include a Power Supply 314. The Power Supply 314 can include a rechargeable or non-rechargeable battery that can be configured to be either user- or non-user-replaceable. In some implementations, the Power Supply 314 can include power-conversion or management circuits (including recharging, standby, or another power management functionality). In some implementations, the Power Supply 314 can include a power plug to allow the Computer 302 to be plugged into a wall socket or another power source to, for example, power the Computer 302 or recharge a rechargeable battery.

There can be any number of Computers 302 associated with, or external to, a computer system containing Computer 302, each Computer 302 communicating over Network 330. Further, the term “client,” “user,” or other appropriate terminology can be used interchangeably, as appropriate, without departing from the scope of the present disclosure. Moreover, the present disclosure contemplates that many users can use one Computer 302, or that one user can use multiple computers 302.

Described implementations of the subject matter can include one or more features, alone or in combination.

For example, in a first implementation, a VR device, comprising: a housing that includes two openings each hosting a camera lens and a nose groove; and one or more cameras distributed around each of the camera lenses for capturing one or more eye physiological characteristics of a VR device user.

The foregoing and other described implementations can each, optionally, include one or more of the following features:

A first feature, combinable with any of the following features, wherein the one or more eye physiological characteristics include iris characteristics or eyeprint characteristics.

A second feature, combinable with any of the previous or following features, further comprising one or more LED light sources for providing light compensation to the one or more cameras.

A third feature, combinable with any of the previous or following features, wherein the one or more LED light sources and the one or more cameras have a one-to-one correspondence.

A fourth feature, combinable with any of the previous or following features, wherein the one or more cameras are infrared cameras and the one or more LED light sources are infrared LED light sources.

A fifth feature, combinable with any of the previous or following features, wherein the one or more cameras are RGB cameras and the one or more LED light sources are RGB LED light sources.

A sixth feature, combinable with any of the previous or following features, wherein the one or more cameras are uniformly distributed around each of the camera lenses.

A seventh feature, combinable with any of the previous or following features, wherein the camera lens is a macro lens.

An eighth feature, combinable with any of the previous or following features, further comprising a data interface including at least a universal serial bus (USB) port or a wireless chip.

A ninth feature, combinable with any of the previous or following features, further comprising a light sensor for sensing the brightness of the light incident on the camera lens.

In a second implementation, a VR system, comprising: a housing of a VR device, wherein the housing includes two openings each hosting a camera lens and a nose groove; and one or more cameras distributed around each of the camera lenses for capturing one or more eye physiological characteristics of a VR device user.

The foregoing and other described implementations can each, optionally, include one or more of the following features:

A first feature, combinable with any of the following features, wherein the one or more eye physiological characteristics include iris characteristics or eyeprint characteristics.

A second feature, combinable with any of the previous or following features, further comprising one or more LED light sources for providing light compensation to the one or more cameras.

A third feature, combinable with any of the previous or following features, wherein the one or more LED light sources and the one or more cameras have a one-to-one correspondence.

A fourth feature, combinable with any of the previous or following features, wherein the one or more cameras are infrared cameras and the one or more LED light sources are infrared LED light sources.

A fifth feature, combinable with any of the previous or following features, wherein the one or more cameras are RGB cameras and the one or more LED light sources are RGB LED light sources.

A sixth feature, combinable with any of the previous or following features, wherein the one or more cameras are uniformly distributed around each of the camera lenses.

A seventh feature, combinable with any of the previous or following features, wherein the camera lens is a macro lens.

An eighth feature, combinable with any of the previous or following features, further comprising a data interface including at least a universal serial bus (USB) port or a wireless chip.

A ninth feature, combinable with any of the previous or following features, further comprising a light sensor for sensing the brightness of the light incident on the camera lens.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs, that is, one or more modules of computer program instructions encoded on a tangible, non-transitory, computer-readable medium for execution by, or to control the operation of, a computer or computer-implemented system. Alternatively, or additionally, the program instructions can be encoded in/on an artificially generated propagated signal, for example, a machine-generated electrical, optical, or electromagnetic signal that is generated to encode information for transmission to a receiver apparatus for execution by a computer or computer-implemented system. The computer-storage medium can be a machine-readable storage device, a machine-readable storage substrate, a random or serial access memory device, or a combination of computer-storage mediums. Configuring one or more computers means that the one or more computers have installed hardware, firmware, or software (or combinations of hardware, firmware, and software) so that when the software is executed by the one or more computers, particular computing operations are performed.

The term “real-time,” “real time,” “realtime,” “real (fast) time (RFT),” “near(ly) real-time (NRT),” “quasi real-time,” or similar terms (as understood by one of ordinary skill in the art), means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second (s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

The terms “data processing apparatus,” “computer,” or “electronic computer device” (or an equivalent term as understood by one of ordinary skill in the art) refer to data processing hardware and encompass all kinds of apparatus, devices, and machines for processing data, including by way of example, a programmable processor, a computer, or multiple processors or computers. The computer can also be, or further include special purpose logic circuitry, for example, a central processing unit (CPU), an FPGA (field programmable gate array), or an ASIC (application-specific integrated circuit). In some implementations, the computer or computer-implemented system or special purpose logic circuitry (or a combination of the computer or computer-implemented system and special purpose logic circuitry) can be hardware- or software-based (or a combination of both hardware- and software-based). The computer can optionally include code that creates an execution environment for computer programs, for example, code that constitutes processor firmware, a protocol stack, a database management system, an operating system, or a combination of execution environments. The present disclosure contemplates the use of a computer or computer-implemented system with an operating system of some type, for example LINUX, UNIX, WINDOWS, MAC OS, ANDROID, IOS, another operating system, or a combination of operating systems.

A computer program, which can also be referred to or described as a program, software, a software application, a unit, a module, a software module, a script, code, or other component can be written in any form of programming language, including compiled or interpreted languages, or declarative or procedural languages, and it can be deployed in any form, including, for example, as a stand-alone program, module, component, or subroutine, for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a communication network.

While portions of the programs illustrated in the various figures can be illustrated as individual components, such as units or modules, that implement described features and functionality using various objects, methods, or other processes, the programs can instead include a number of sub-units, sub-modules, third-party services, components, libraries, and other components, as appropriate. Conversely, the features and functionality of various components can be combined into single components, as appropriate. Thresholds used to make computational determinations can be statically, dynamically, or both statically and dynamically determined.

Described methods, processes, or logic flows represent one or more examples of functionality consistent with the present disclosure and are not intended to limit the disclosure to the described or illustrated implementations, but to be accorded the widest scope consistent with described principles and features. The described methods, processes, or logic flows can be performed by one or more programmable computers executing one or more computer programs to perform functions by operating on input data and generating output data. The methods, processes, or logic flows can also be performed by, and computers can also be implemented as, special purpose logic circuitry, for example, a CPU, an FPGA, or an ASIC.

Computers for the execution of a computer program can be based on general or special purpose microprocessors, both, or another type of CPU. Generally, a CPU will receive instructions and data from and write to a memory. The essential elements of a computer are a CPU, for performing or executing instructions, and one or more memory devices for storing instructions and data. Generally, a computer will also include, or be operatively coupled to, receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a global positioning system (GPS) receiver, or a portable memory storage device.

Non-transitory computer-readable media for storing computer program instructions and data can include all forms of permanent/non-permanent or volatile/non-volatile memory, media and memory devices, including by way of example semiconductor memory devices, for example, random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices; magnetic devices, for example, tape, cartridges, cassettes, internal/removable disks; magneto-optical disks; and optical memory devices, for example, digital versatile/video disc (DVD), compact disc (CD)-ROM, DVD+/−R, DVD-RAM, DVD-ROM, high-definition/density (HD)-DVD, and BLU-RAY/BLU-RAY DISC (BD), and other optical memory technologies. The memory can store various objects or data, including caches, classes, frameworks, applications, modules, backup data, jobs, web pages, web page templates, data structures, database tables, repositories storing dynamic information, or other appropriate information including any parameters, variables, algorithms, instructions, rules, constraints, or references. Additionally, the memory can include other appropriate data, such as logs, policies, security or access data, or reporting files. The processor and the memory can be supplemented by, or incorporated in, special purpose logic circuitry.

To provide for interaction with a user, implementations of the subject matter described in this specification can be implemented on a computer having a display device, for example, a CRT (cathode ray tube), LCD (liquid crystal display), LED (Light Emitting Diode), or plasma monitor, for displaying information to the user and a keyboard and a pointing device, for example, a mouse, trackball, or trackpad by which the user can provide input to the computer. Input can also be provided to the computer using a touchscreen, such as a tablet computer surface with pressure sensitivity, a multi-touch screen using capacitive or electric sensing, or another type of touchscreen. Other types of devices can be used to interact with the user. For example, feedback provided to the user can be any form of sensory feedback (such as, visual, auditory, tactile, or a combination of feedback types). Input from the user can be received in any form, including acoustic, speech, or tactile input. In addition, a computer can interact with the user by sending documents to and receiving documents from a client computing device that is used by the user (for example, by sending web pages to a web browser on a user's mobile computing device in response to requests received from the web browser).

The term “graphical user interface,” or “GUI,” can be used in the singular or the plural to describe one or more graphical user interfaces and each of the displays of a particular graphical user interface. Therefore, a GUI can represent any graphical user interface, including but not limited to, a web browser, a touch screen, or a command line interface (CLI) that processes information and efficiently presents the information results to the user. In general, a GUI can include a number of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server, or that includes a front-end component, for example, a client computer having a graphical user interface or a Web browser through which a user can interact with an implementation of the subject matter described in this specification, or any combination of one or more such back-end, middleware, or front-end components. The components of the system can be interconnected by any form or medium of wireline or wireless digital data communication (or a combination of data communication), for example, a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11 a/b/g/n or 802.20 (or a combination of 802.11x and 802.20 or other protocols consistent with the present disclosure), all or a portion of the Internet, another communication network, or a combination of communication networks. The communication network can communicate with, for example, Internet Protocol (IP) packets, Frame Relay frames, Asynchronous Transfer Mode (ATM) cells, voice, video, data, or other information between network nodes.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of any invention or on the scope of what can be claimed, but rather as descriptions of features that can be specific to particular implementations of particular inventions. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results. In certain circumstances, multitasking or parallel processing (or a combination of multitasking and parallel processing) can be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

What is claimed is:

1. A virtual reality (VR) device, comprising:
a housing that includes two openings each hosting a camera lens and a nose groove; and
one or more cameras distributed around each of the camera lenses for capturing one or more eye physiological characteristics of a VR device user.

2. The VR device of claim 1, wherein the one or more eye physiological characteristics include iris characteristics or eyeprint characteristics.

3. The VR device of claim 1, further comprising one or more light emitting diode (LED) light sources for providing light compensation to the one or more cameras.

4. The VR device of claim 3, wherein the one or more LED light sources and the one or more cameras have a one-to-one correspondence.

5. The VR device of claim 3, wherein the one or more cameras are infrared cameras and the one or more LED light sources are infrared LED light sources.

6. The VR device of claim 3, wherein the one or more cameras are RGB cameras and the one or more LED light sources are RGB LED light sources.

7. The VR device of claim 1, wherein the one or more cameras are uniformly distributed around each of the camera lenses.

8. The VR device of claim 1, wherein the camera lens is a macro lens.

9. The VR device of claim 1, further comprising a data interface including at least a universal serial bus (USB) port or a wireless chip.

10. The VR device of claim 1, further comprising a light sensor for sensing the brightness of the light incident on the camera lens.

11. A virtual reality (VR) system, comprising:
a housing of a VR device, wherein the housing includes two openings each hosting a camera lens and a nose groove; and
one or more cameras distributed around each of the camera lenses for capturing one or more eye physiological characteristics of a VR device user.

12. The VR system of claim 11, wherein the one or more eye physiological characteristics include iris characteristics or eyeprint characteristics.

13. The VR system of claim 11, further comprising one or more light emitting diode (LED) light sources for providing light compensation to the one or more cameras.

14. The VR system of claim 13, wherein the one or more LED light sources and the one or more cameras have a one-to-one correspondence.

15. The VR system of claim 13, wherein the one or more cameras are infrared cameras and the one or more LED light sources are infrared LED light sources.

16. The VR system of claim 13, wherein the one or more cameras are RGB cameras and the one or more LED light sources are RGB LED light sources.

17. The VR system of claim 11, wherein the one or more cameras are uniformly distributed around each of the camera lenses.

18. The VR system of claim 11, wherein the camera lens is a macro lens.

19. The VR system of claim 11, further comprising a data interface including at least a universal serial bus (USB) port or a wireless chip.

20. The VR system of claim 11, further comprising a light sensor for sensing the brightness of the light incident on the camera lens.
